(19) United States
(12) Patent Application Publication
(10) Pub. No.: US 2006/0047301 A1
(43) Pub. Date: Mar. 2, 2006

(54) Emboli removal system with oxygenated flow

(76) Inventor: Matthew F. Ogle, Oronoco, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/216,186

(22) Filed: Aug. 31, 2005

Related U.S. Application Data
(60) Provisional application No. 60/606,844, filed on Sep. 2, 2004.

(51) Int. Cl. A61M 29/00

(52) U.S. Cl. 606/200; 604/43

(57) ABSTRACT

Medical treatment systems incorporate inflow catheters to delivery oxygenated blood into a blood vessel. Devices suitable for the removal of emboli can be used with the inflow catheters. Suitable emboli removal structures include, for example, aspiration catheters and/or vascular filters. Blood removed with aspiration catheters can be filtered and returned to the patient through the inflow catheter.

### CROSS REFERENCE TO RELATED APPLICATIONS

This application claims priority to copending provisional patent application 60/606,844 filed on Sep. 2, 2004 to Ogle, entitled “Emboli Removal System With Oxygenated Backflow,” incorporated herein by reference.

### FIELD OF THE INVENTION

The present invention relates to medical systems designed to remove emboli, such as with suction from a patient's vessel, and also providing oxygenated backflow downstream. In some embodiments, the medical systems further comprise a filter within the vessel. Also, the invention pertains to procedures for providing oxygenated backflow while performing intervention within a blood vessel.

### BACKGROUND OF THE INVENTION

Distal capillary beds perform an important role in the perfusion of oxygenated blood within tissue and organs. This role can have implications with respect to recent advanced techniques in interventional vascular therapies. Advancements in a wide range of intervention technologies have called attention to the phenomenon of distal embolization of particulate matter. Specifically, microembolization can occur during a coronary intervention procedure, such as the deployment of a balloon or a stent to open a restriction within a vessel at a lesion. The intervention procedure itself can liberate pieces of thrombus or atheroma from the lesion. The released particulate debris can then migrate downstream and lodge in distal capillary beds. The obstructions resulting from embolization of the distal capillary beds potentially result in areas of microinfarctions and necrosis. Microembolization has been shown to have negative impact on clinical outcomes and survival. The consequences depend on the number and size of emboli and on the sensitivity of the organ.

Similarly, injuries, other trauma or general degeneration of a portion of the vascular system can result in an increased chance of emboli formation in the affected section of blood vessel. These emboli can travel to distal locations at which flow can be blocked as a result of an embolization. These embolization events can lead to serious consequences if they take place in sensitive organs, such as the heart, lungs or brain, due to travel of the emboli from the injury site.

### SUMMARY OF THE INVENTION

In some aspects, the invention pertains to an emboli flushing system comprising an oxygenated blood supply, blood delivery apparatus and an inflow catheter attached to the blood supply to deliver blood through the catheter through the action of the blood delivery apparatus. In some embodiment, the emboli flushing system further comprises an aspiration catheter comprising a catheter and a suction device, in which the aspiration catheter has an appropriate size for use with the inflow catheter. The aspiration catheter can interface with the inflow catheter with a rapid exchange configuration or with an over-the-wire type configuration. The inflow from the aspiration catheter can be filtered and returned through the inflow catheter. This filtering can be done within the blood vessel or external to the patient.

In further aspects, the invention pertains to a method for collecting emboli, the method comprising infusing blood within a blood vessel to create a backflow within the blood vessel and aspirating excess blood from the vessel relating to the backflow. In some embodiments, the method further comprises filtering the blood in the backflow to remove emboli and/or filtering blood aspirated from the vessel. The aspirated blood can be returned to the blood vessel following filtering to remove emboli.

In an additional aspect, the invention pertains to a medical treatment system comprising an aspiration catheter and a profusion catheter. The aspiration catheter can comprises a suction device and a tube with an aspiration lumen and a distal aspiration port. The distal aspiration port comprises an opening at the distal tip of the catheter. The aspiration lumen is in fluid communication with the suction device. The profusion catheter comprises a reservoir of oxygenated blood and a tube having a profusion lumen and a profusion port providing an exit from the profusion lumen at or near the distal end of the profusion lumen. The profusion lumen is in fluid communication with the reservoir of oxygenated blood. The profusion catheter and the aspiration catheter generally are configured such that the profusion catheter can be deployed within a vessel of a patient with the profusion opening downstream from the distal aspiration opening.

In other aspects, the invention pertains to a medical treatment system comprising a profusion catheter and a filter device. The profusion catheter comprises a reservoir of oxygenated blood and a tube having a profusion lumen and a profusion port providing an exit from the profusion lumen at or near the distal end of the profusion lumen. The profusion lumen is in fluid communication with the reservoir of oxygenated blood. The filter device can have a low profile configuration and an extended configuration. Also, the filter device can be configured on a delivery catheter for delivery over the profusion catheter within the vessel of a patient.

Moreover, the invention pertains to a method for delivering oxygenated blood to distal capillary beds. The method comprises delivering oxygenated blood within a blood vessel from a reservoir external to the patient in which the oxygenated blood is delivered from an inflow catheter positioned with an inflow port downstream from an emboli intervention device.

### BRIEF DESCRIPTION OF THE DRAWINGS

FIG. 1 is a side view of an embodiment of an emboli-flushing system.

FIG. 2 is a fragmentary side view of an embodiment of an emboli flushing system in which a pump directs fluid from an aspiration catheter to a inflow catheter.

FIG. 3 is a fragmentary side view of an alternative embodiment of an inflow catheter shown positioned within a blood vessel along with a treatment device and a filter downstream from the treatment structure.

FIG. 4A is a fragmentary sectional view depicting the fiber based filter of FIG. 3 with the section taken through the center of the catheter.

FIG. 4B is a sectional view of the fiber based filed taken along line B-B of FIG. 4A.

FIG. 5 is a fragmentary side view of an inflow catheter positioned within a blood vessel along with a treatment device and a filter upstream from the treatment structure.

### DETAILED DESCRIPTION OF THE INVENTION

As described herein, an effective approach for the prevention of deleterious effects of emboli in distal capillary beds and the removal of emboli before they reach distal capillary beds involves the delivery of oxygenated blood into the vascular system. In some embodiments, the oxygenated blood is delivered into the vessel at sufficient flow rates to induce a backflow within the vessel, generally an artery. The back flowing blood can be aspirated from the vessel. If desired, the aspirated blood can be filtered with respect to emboli and subsequently returned to the blood vessel or to a different blood vessel. Alternatively, donor blood or the patient's own pre-stored blood can be delivered to the patient. In some embodiments, the oxygenated blood is delivered at a rate comparable to or less than the natural flow rate such that the conveyed blood replaces temporarily blocked blood flow and/or lessens the upstream flow rate at points of intervention. The oxygenated blood can be delivered through a catheter with an appropriate diameter at a corresponding appropriate rate. The tip of the inflow catheter can be located appropriately during delivery of the oxygenated blood, for example, between distal capillary beds and a lesion or an injury. The catheter device can be used in conjunction with an aspiration catheter to remove the excess blood flow, as well as optionally with a vascular filter device and/or an intervention device, such as a balloon and/or a stent.

In general, less invasive techniques can be used to treat blockages and/or injuries to blood vessels using catheters, guidewires and the like to deliver treatment structures to the lesion. Thus, intervention approaches themselves can generate emboli that can flow downstream to threaten blockage of small vessels, such as capillaries. Approaches can be used to capture these emboli to prevent or reduce the risk from emboli. However, these approaches may not be fully effective and/or these approaches can disrupt the natural flow in the vessel. The delivery of oxygenated blood with an inflow catheter can ameliorate the effects of natural flow blockage, can induce a backflow that pushes the emboli upstream for removal and/or can slow the passage of emboli down stream so that they can be captured more readily with approaches intended to capture the emboli before they escape downstream. Suitable patients include human as well as farm animals, pets and other mammals. In general, the procedures are suitable for any blood vessels, and coronary arteries and the carotid artery are blood vessels of specific interest.

With respect to approaches for the removal of emboli from the vessel, these approaches can involve, for example, aspiration and/or filters. Aspiration catheters can be used to remove emboli from the vessel downstream from the inflow catheter. Alternatively or additionally, an approach for the prevention of vascular obstructions from emboli involves the placement of vascular filters distal to a point of intervention or trauma. Removal of the vascular filters from the vessel can correspondingly remove the trapped emboli from the vessel.

Highly effective filter designs based on three dimensional filtering matrix have been developed. Some of these filters are based on fibers, such as surface capillary fibers. These filtering devices are described further in copending U.S. patent applications Ser. Nos. 10/414,909 to Ogle, entitled “Embolism Protection Devices,” 10/806,311 to Ogle, entitled “Embolism Protection Devices,” and 10/795,131 to Ogle et al, entitled “Fiber Based Embolism Protection Device,” all three of which are incorporated herein by reference. In some embodiments, vascular filters, such as the improved filters with a three-dimensional filtering matrix, can be combined with the inflow catheter for backflow flushing of emboli, although the appropriate location for filter placement may be changed in a backflow situation, as described further below. Other filter systems using baskets or the like can be used, and some are commercially available. Occlusive elements, such as balloons and webs can be used to temporarily restrict flow, for example to aspirate the region to remove emboli.

With respect to the delivery of oxygenated blood into a patient's vessel, an inflow catheter can be used generally in conjunction with other devices. The inflow catheter can be connected to an oxygenated blood source. The blood can originate from the patient or from a suitable donor. In some embodiments, the system further comprises an aspiration catheter that removes blood and any emboli from an appropriate position proximal to the tip of the inflow catheter for the removal of excess blood from the vessel as a result of the inflow. The system can further be used in combination with an intervention device and/or a filter, such as a vascular filter with a three dimensional matrix and/or a blood filter used external to the patient.

The inflow catheter generally has a small diameter such that the catheter can be placed at an appropriate location within the vessel, while allowing for the placement of additional instruments within the vessel over the inflow catheter. The inflow catheter can have a blood compatible coating on the inner surface, such as polytetrafluoroethylene, to reduce any damage to the blood components flowing through the catheter. In general, the inflow catheter can be formed form suitable biocompatible and blood compatible materials, such as metal, polymers or a combination thereof. For example, the catheter can be formed from a polymer tube, such as polyamides, polyolefins, polyesters, polyurethanes, polycarbonates, combinations thereof or other biocompatible polymers or blends. Metal wire, such as stainless steel or alloys, e.g., nickel titanium alloys, can be embedded in a polymer catheter to influence the catheter properties. The pumping of the blood through the catheter can be continuous or pulsed. If pulsed, the pulses of pumping through the catheter can be timed to approximately coincide with the patient's pulse. The tip of the inflow catheter can be designed to reduce the turbulence or other undesirable flow properties resulting from the mixing of the blood from the catheter with the flow in the vessel. For example, the tip of the catheter can have an unrestrained opening or an expanded opening and/or side ports to facilitate the exit of the blood from the catheter.

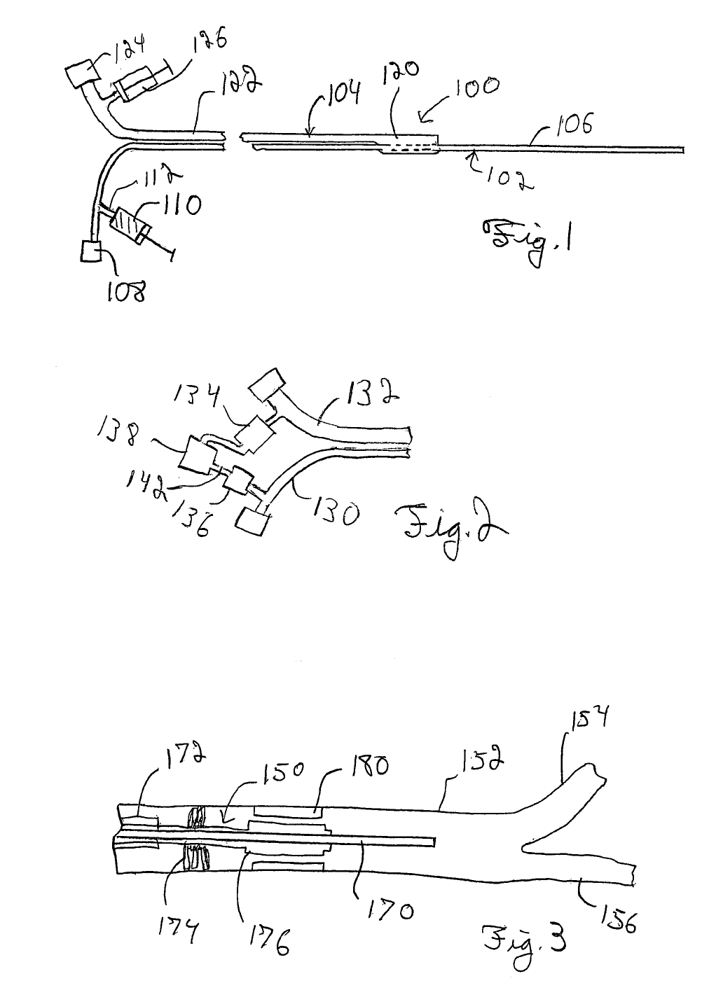

Referring to FIG. 1, one embodiment of an inflow blood delivery system 100 is shown comprising an inflow catheter 102 and an aspiration catheter 104. Inflow catheter 102 comprises a tubular section 106, proximal section 108 and oxygenated blood source 110. Tubular section 106 can have a generally constant diameter over the blood flow portion, or the diameter can vary in a selected pattern. Proximal section 108 can comprise a handle, ports or other convenient control structures. Oxygenated blood source 110 comprises a blood delivery apparatus, such as a syringe, a pump or the like, along with a reservoir of oxygenated blood. However, in some embodiments, a reservoir of oxygenated blood can be replaced or supplemented with a continuous source of oxygenated blood such as the aspiration catheter, a hypodermic needle in the patient or a blood oxygenator with a supply of suitable blood. Oxygenated blood source 110 can be connected to tubular section 106 at the proximal section 108 or at a separate connection 112 that may have a suitable fitting, such as a rubber septum or a Luer lock.

Aspiration catheter 104 can comprise a distal rapid exchange section 120, a tubular section 122, a proximal end 124 and a suction apparatus 126. Rapid exchange section 120 comprises a port through which tubular section 106 of inflow catheter 102 can be passed. While a rapid exchange section can be convenient for using device 100, other embodiments have an aspiration catheter that passes over the inflow catheter roughly along its entire length. Tubular section 120 can have an approximately constant diameter or the diameter can vary according to appropriate designs based on desired suction into the catheter. Proximal end 124 can comprise a handle, ports and/or other appropriate control structures. Suction apparatus 126 can comprise a syringe, pump or the like. Suction apparatus 126 can be connected to tubular section 120 at proximal end 124 or at a separate connection 128 that may have a suitable fitting, such as a Luer lock. Aspiration catheters for emboli removal are described further in copending U.S. patent application Ser. No. 10/854,920 to Pokorney et al., entitle “Emboli Export Catheter,” and copending U.S. patent application Ser. No. 11/207,169 on Aug. 18, 2005 to Boldenow et al., entitled “Improved Tracking Aspiration Catheter,” both of which are incorporated herein by reference.

Referring to FIG. 2, an inflow blood delivery system has an inflow catheter 130 and an aspiration catheter 132. Aspiration catheter 132 comprises a collection reservoir 134, and inflow catheter 130 comprises an oxygenated blood reservoir 136. Pump 138 pumps blood between collection reservoir 134 and oxygenated blood reservoir 136. An optional filter 142 can be placed at a suitable place on the flow path from the aspiration catheter lumen and the inflow catheter lumen. Pump 138 can provide the suction for the aspiration catheter and/or positive pressure for the inflow of blood into the inflow catheter, or a separate suction device and/or oxygenated blood pump can be used to control flow between the respective reservoirs and the respective catheter lumen.

Referring to a fragmentary view in FIG. 3, an inflow blood delivery system 150 is depicted within a blood vessel 152 that branches into vessels 154 and 156. Inflow system 150 comprises an inflow catheter 170, an aspiration catheter 172, a vascular filter 174 and an intervention device 176. In this embodiment, the inflow catheter is used in conjunction with an optional filter and an optional intervention device. Through the use of a vascular filter, the blood entering aspiration catheter 172 has been filtered to remove at least a portion of the emboli. Suitable vascular filters include, for example, vascular filters with a three dimensional filtering matrix, which can comprise fibers or the like, as described in more detail above. A shown in FIG. 3, intervention device 176 is positioned at a constriction 180 in blood vessel 152, although the device can be used to treat other types of lesions. Intervention device 176 can comprise an angioplasty balloon, a stent or the like. Vascular filter 174 can be removed using the aspiration catheter as a sheath. The removal of vascular filters using an aspiration catheter is described further in copending U.S. patent application Ser. No. 10/854,920 to Pokorney et al., entitle “Emboli Export Catheter,” incorporated herein by reference.

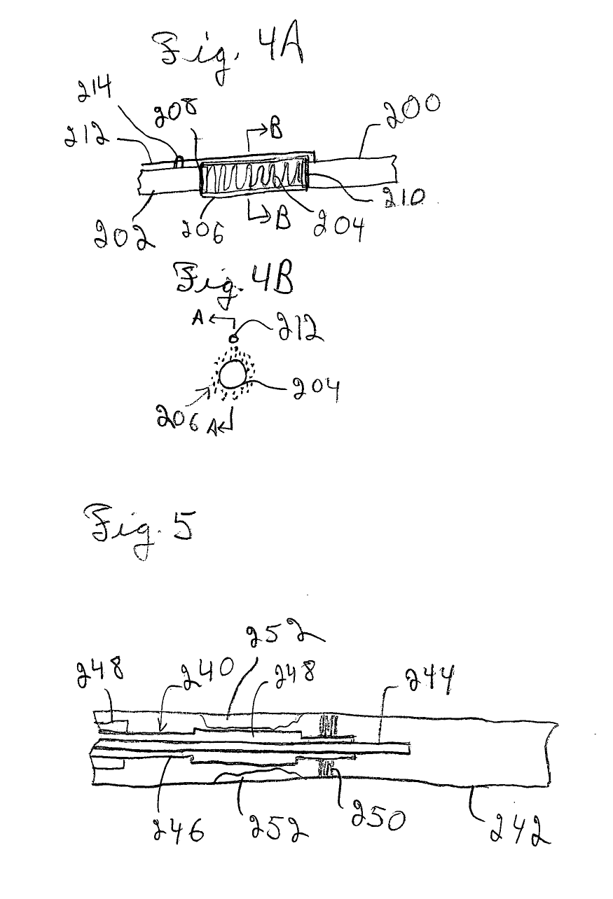

With respect to fiber based filters, an embodiment suitable for use in the system of FIG. 3 is shown in FIG. 4A in a low profile configuration for delivery into the vessel. This device comprises a distal tubular portion 200, a proximal tubular portion 202, a spring 204 connecting the distal tubular portion 200 and the proximal tubular portion 202, a bundle of fibers 206, fiber anchors 208, 210, an actuation wire 212 and a wire guide 214. The fiber bundle can be seen in the sectional view of FIG. 4B. Fiber anchors 208, 210 can comprise metal bands, adhesive, combinations thereof or the like to fix the ends of the fibers. Actuation wire 212 can be used to draw distal tubular portion 200 closer to proximal tubular portion 202 to compress spring 204 and cause fibers 206 to flair outward into their deployed/expanded configuration. Similarly, a plurality of actuation wires, such as two, three, four or more, can be used to draw distal tubular portion 200 closer to proximal tubular portion 202. If a plurality of action wires are used, these can be distributed approximately symmetrically about the circumference of the catheter to apply corresponding approximately symmetrical forces, and a plurality of wire guides can be correspondingly used. Wire guide 214 can be one or more eyelets, a wire lumen or the like. If a fluid lumen extends from the proximal portion of the catheter to the therapy device 176 past a filter as shown in FIG. 4A, the fluid lumen can comprise nested tubular portions with one portion sliding within the other portion to account for the shortened length in the deployed configuration with spring 204. An o-ring, washer or the like can prevent leaks upon movement of the nested tubes.

An alternative embodiment of an inflow oxygenated blood delivery system 240 is shown in a fragmentary view in FIG. 5 within a patient's vessel 242. System 240 comprises an inflow catheter 244, an intervention catheter 246 and an aspiration catheter 248. Intervention catheter 246 comprises an intervention structure 248, such as a balloon, a stent, or the like, and a filter 250. In this embodiment, filter 250 is distal or downstream from intervention structure 248. Thus, filter 250 can collect emboli generated from the use of intervention structure 248 for the treatment of lesion 252. Thus, if inflow catheter 244 does not have sufficient flow to generate a backflow, filter 250 can reduce or eliminate migration of emboli downstream. Filter 250 can have the structure as shown in FIG. 4A or other reasonable structure. In further embodiments, filters can be placed both downstream from an intervention structure, as shown in FIG. 5, and upstream, as shown in FIG. 3.

The systems described herein can be used for backflow flushing of emboli. However, the systems can also be suitable for profusing downstream capillaries with oxygenated blood without actually generating a backflow. If lesser flows are used, the delivery of oxygenated blood can slow the movement of emboli upstream to make their capture and/or removal easier.

With respect to the backflow flushing of emboli, an emboli-flushing system can provide a blood inflow that delivers sufficient blood within a vessel to induce a backflow in the vessel proximal to the tip of the catheter. The inflow catheter can be used to generate a backflow in a vessel susceptible to emboli to flush any emboli upstream relative to the natural flow for removal, such as through an aspiration catheter. Thus, with the use of an inflow catheter and an aspiration catheter a section of blood vessel can be flushed without interrupting the flow of oxygenated blood to distal capillaries. The flushing removes blood that can contain emboli, which may or may not be filtered and returned to the patient.

For these backflow embodiments, the flow rate through the inflow catheter is correspondingly higher than the flow rate through the corresponding vessel. Since the diameter of the vessel is larger than the diameter of the catheter, the velocity of the blood in the catheter is correspondingly higher. An excess flow rate through the catheter is the difference between the natural flow rate through the vessel and the total flow rate through the inflow catheter. The excess flow is beyond the amount of fluid required to supply the distal portion of the vessel and results in the backflow. The amount of backflow can be selected as desired. The suction applied with the aspiration catheter generally can remove a flow corresponding approximately equal with the total flow from the in-flow catheter. This total flow going into the aspiration catheter effectively includes the excess flow that results in the backflow from the inflow catheter as well as the natural downstream flow through the vessel. The aspiration catheter can be designed for delivery over the in-flow catheter (with an over-the-wire configuration or a rapid exchange configuration, as shown in FIG. 1) or for deployment adjacent the inflow catheter. The aspiration can be applied with a syringe, a pump or the like.

In some embodiments, the fluid removed with the aspiration catheter can be filtered and returned through the inflow catheter. This filtering can be performed within the vessel, within the catheter and/or external to the vessel and catheter. Filtering within the vessel can be performed, for example, using a device as shown in FIG. 3. Similarly, a suitable filter can be placed within the suction lumen of the aspiration catheter. In some embodiments, a filter can be placed at the collection vessel for the collected blood or in a flow path between the aspiration catheter and the inflow catheter, as shown in FIG. 2.

The flushing system with the inflow catheter and the aspiration catheter are designed to flush flow within the vessel roughly between the inflow port, which can be located at or near the tip of the inflow catheter, and the suction port, which can be at or near the tip of the aspiration catheter. The flushed portion of the vessel can include, for example, an injured portion of the vessel or a lesion, such as a location of atherosclerotic build-up, that undergoes intervention, for example, with a balloon angioplasty or a stent placement.

If the system is used in the case of an injury, the inflow catheter and aspiration catheter can be used for a significant period of time, for example, until the traumatized vessel stabilizes as a result of natural healing properties. In some embodiments, the traumatized vessel can be treated for several hours, in some embodiments for a day or more and in further embodiments for a week or more. In these embodiments, it generally is desirable to re-circulate blood from the aspiration catheter to the in-flow catheter following filtering. This filtering can be performed external to the body since a suitable re-circulation pumping network can be established and/or internal to the body with a vascular filter if the embolic load is appropriate for the filter capacity.

If the system is used at a point of treatment/intervention, the backflow flushing of the intervention point can be established a short time prior to the intervention. Once in place, the treatment at the lesion can be applied. Any emboli generated during the intervention are caught in the backflow and thereby prevented from flowing into distal capillary beds. Any emboli generated within the backflow can be removed through the aspiration catheter or filter within the vessel using a filter placed within the vessel proximal to the lesion, i.e., upstream with respect to the natural flow within the vessel. In this configuration, the filter does not need to be inserted past the lesion. The filter can be removed once the intervention is completed. The removal of a filter with a three dimensional filtration matrix is described in copending patent application Ser. No. 10/854,920 to Galdonik et al., entitled “Emboli Filter Export System,” incorporated herein by reference. If a filter is used within the vessel, it may be less likely for emboli to get trapped within the vessel. In these embodiments, the aspirated flow can be returned to the in-flow catheter without additional filtering, although additional filtration can also be performed on the returned flow as an added precaution. In these or any other embodiments, blood can be collected from the patient prior to the procedure such that the patient can be confident of the suitability of the blood, although in some embodiments donor blood can be used. While pre-collected blood can be used exclusively in the inflow catheter, in some embodiments, pre-collected blood can supplement any blood recirculated between the aspiration catheter and the inflow catheter.

In some embodiments, lower flow rates can be used advantageously without generating a backflow. In these embodiments, the oxygenated blood flow can replace temporarily blocked flow or reduced flow without eliminating downstream flow. For example, using the device in FIG. 3 with or without the filter, the therapy device can be expanded and kept expanded for sufficient time to aspirate the vessel to remove any emboli generated downstream from the lesion. The aspiration can be continued following deflation of the treatment structure to remove additional emboli. These approaches can be useful in situation in which there is a high embolic load generated from the therapy such that a filter may not be best as used without other embolic removal approaches. While all emboli may not be removed through this approach, the inflow facilitates removal of emboli and improves the collection. If desired, a filter can also be used downstream from the treatment structure, as shown in FIG. 5. The profusion of blood through the inflow catheter lowers the blood pressure at the filter and any aspiration performed before emboli reach the filter decrease the emboli load on the filter.

The systems and methods described herein can be used to offer a range of alternative treatment approaches. Through providing oxygenated blood, a treating physician can use alternative treatment approaches that reduce flow during the treatment process more than desirable without the supply of oxygenated blood or that could produce more emboli than can be safely handled without the introduction of oxygenated blood to assist with the procedure.

The embodiments above are intended to be illustrative and not limiting. Additional embodiments are within the inventive concepts. Although the present invention has been described with reference to particular embodiments, workers skilled in the art will recognize that changes may be made in form and detail without departing from the spirit and scope of the invention. Any incorporation by reference of documents above is limited so that no subject matter is incorporated that is contrary to the explicit disclosure herein.

What is claimed is:

1. A medical treatment system comprising:
an aspiration catheter comprising a suction device and a tube with an aspiration lumen and a distal aspiration port, the aspiration lumen being in fluid communication with the suction device; and
a profusion catheter comprising a reservoir of oxygenated blood and a tube with a profusion lumen and a profusion port providing an exit from the profusion lumen at or near the distal end of the profusion lumen, the profusion lumen being in fluid communication with the reservoir of oxygenated blood,
wherein the profusion catheter and the aspiration catheter are configured such that the profusion catheter can be deployed within a vessel of a patient with the profusion opening downstream from the distal aspiration opening.

2. The medical treatment system of claim 1 wherein the aspiration catheter has a rapid exchange port.

3. The medical treatment system of claim 1 wherein the suction device comprises a syringe.

4. The medical treatment system of claim 1 further comprising a pump providing fluid communication between the suction device and the reservoir of oxygenated blood.

5. The medical treatment system of claim 1 wherein the profusion catheter further comprises a pump configured to deliver oxygenated blood from the reservoir to the profusion lumen.

6. The medical treatment system of claim 1 wherein the aspiration catheter connects with a portion of the profusion catheter such that at least over a portion of their length, the aspiration catheter and the profusion catheter form an integral structure.

7. The medical treatment system of claim 6 wherein a distal portion of the profusion catheter passes through a rapid exchange port of the aspiration catheter to provide the connection between the aspiration catheter and the profusion catheter.

8. A medical treatment system comprising:
a profusion catheter comprising a reservoir of oxygenated blood and a tube with a profusion lumen and a profusion opening providing an exit from the profusion lumen at or near the distal end of the profusion lumen, the profusion lumen being in fluid communication with the reservoir of oxygenated blood; and
a filter device having a low profile configuration and an extended configuration, wherein the filter device is configured on a delivery catheter for delivery over the profusion catheter within the vessel of a patient.

9. The medical treatment system of claim 8 wherein the filter device comprises polymer fibers.

10. The medical treatment system of claim 9 wherein the polymer fibers comprise surface capillary fibers.

11. The medical treatment system of claim 8 wherein a vascular treatment structure is mounted on the delivery catheter at a distal position relative to the filter device.

12. The medical treatment system of claim 11 wherein the vascular treatment structure comprises a balloon.

13. The medical treatment system of claim 11 wherein the vascular treatment structure comprises a stent.

14. The medical treatment system of claim 11 wherein a vascular treatment structure is mounted on the delivery catheter at a proximal position relative to the filter device.

15. The medical treatment structure of claim 8 further comprising an aspiration catheter that is configured for delivery within a patient's vessel at least a portion of which is over the delivery catheter, the aspiration catheter comprising a shaft with an aspiration lumen and a suction device in fluid communication with the aspiration lumen.

16. The medical treatment system of claim 8 wherein the delivery catheter interfaces with the profusion catheter in a rapid exchange configuration.

17. A method for delivering oxygenated blood to distal capillary beds, the method comprising delivering oxygenated blood within a blood vessel from a reservoir external to the patient wherein the oxygenated blood is delivered from an inflow catheter positioned with an inflow port downstream from an emboli intervention device.

18. The method of claim 17 wherein the emboli intervention device comprises a filter.

19. The method of claim 17 wherein the emboli intervention device comprises an aspiration catheter.

20. The method of claim 17 further comprising intervening at a lesion with a balloon or a stent while delivering the oxygenated blood.
